(19) United States
(12) Patent Application Publication
(10) Pub. No.: US 2024/0409945 A1
(43) Pub. Date: Dec. 12, 2024

(54) RNAi TARGETING PPP2R5D MISSENSE MUTATIONS FOR TREATMENT OF JORDAN’S SYNDROME

(71) Applicant: Ovid Therapeutics Inc., New York, NY (US)

(72) Inventor: Noelle Germain, Watertown, CT (US)

(21) Appl. No.: 18/735,613

(22) Filed: Jun. 6, 2024

Related U.S. Application Data
(60) Provisional application No. 63/603,863, filed on Nov. 29, 2023.
(60) Provisional application No. 63/506,463, filed on Jun. 6, 2023.

(51) Int. Cl. C12N 15/113

(52) U.S. Cl. CPC C12N 15/1137; C12N 2310/11; C12N 2310/122; C12N 2310/14; C12N 2310/341; C12Y 301/03016

(57) ABSTRACT

RNAi involving siRNA, shRNA or ASO is provided that reduces expression of toxic PPP2R5D alleles containing one or more of E197K, E198K, E200K and E420K missense mutations thereby reducing production of mutant PPP2R5D for treatment of Jordan's Syndrome.

### CROSS REFERENCE TO RELATED APPLICATION

This application claims benefit of and priority to U.S. Provisional Application No. 63/506,463, filed on Jun. 6, 2023, and U.S. Provisional Application No. 63/603,863, filed on Nov. 29, 2023, and which are both incorporated herein by reference in their entireties.

### TECHNICAL FIELD

The present disclosure relates to RNAi compositions that reduce expression of toxic PPP2R5D alleles in the treatment of Jordan's Syndrome.

### REFERENCE TO SEQUENCE LISTING

The application contains a Sequence Listing which has been submitted electronically in .XML format and is hereby incorporated by reference in its entirety. Said. XML copy, created on Jun. 4, 2024, is named “2262-106.xml” and is 166,151 bytes in size. The sequence listing contained in this .XML file is part of the specification and is hereby incorporated by reference herein in its entirety.

### BACKGROUND

Jordan's Syndrome results from de novo mutations to the PPP2R5D gene and occurs with a prevalence of 3/100,000. The phenotype consists of neurodevelopmental delay and intellectual disability, hypotonia, megalencephaly, coordination disorder, seizures, and autism. Affected adults in their 30's to 50's develop Parkinsonism and die of a neurodegenerative condition. PPP2R5D encodes the B56 regulatory subunit of protein phosphatase-2A (PP2A), an intracellular serine/threonine phosphatase involved in several cell signaling pathways implicated in cell growth and differentiation. There are approximately 16 different de novo heterozygous missense alleles associated with this condition, the most frequent of which, Glu198Lys, accounts for about 43% of currently known patients. The hypothesized molecular mechanism underlying this condition is that mutations in PPP2R5D result in altered PPP2R5D substrate specificity, leading to an abnormal phosphoproteomic profile with aberrant phosphorylation of many neural target proteins.

There is currently no effective therapy or cure for Jordan's Syndrome.

### SUMMARY

Provided herein are novel RNA-targeting oligonucleotides, i.e., RNAi involving small interfering RNA (siRNA), short hairpin RNA (shRNA), and antisense oligonucleotides (ASO) that reduce expression of toxic PPP2R5D alleles thereby reducing production of mutant PPP2R5D for treatment of Jordan's Syndrome.

Accordingly, provided herein are RNAi sequences that target one or more of four individual PPP2R5D missense mutations (E197K, E198K, E200K and E420K) that are causative of Jordan's Syndrome. The RNAi sequences can be delivered as short interfering RNA (siRNA) duplexes or transcribed as short hairpin RNA (shRNA) from plasmid DNA. Expression vectors encoding the RNAi sequences are provided. In embodiments, the expression vector is an adeno-associated viral (AAV) vector or a lentiviral vector. In embodiments, siRNA is delivered by nanoparticulate vehicles or by polymeric vehicles. Pharmaceutical compositions including the foregoing are provided.

In embodiments, provided herein are siRNAs targeting individual PPP2R5D missense mutations including a nucleotide sequence that is at least 85%, at least 90%, at least 95%, or 100% complementary to any of SEQ ID NOs: 1-58.

In embodiments, shRNAs are provided having a first portion, a second portion and a third portion, the first portion including SEQ ID NOs: 1-29, the second portion including SEQ ID NOs: 59-66, and the third portion including SEQ ID NOs: 30-58.

Expression vectors including the siRNAs, shRNAs or ASOs are provided. In embodiments, the expression vector is an adeno-associated viral (AAV) vector or a lentiviral vector.

Pharmaceutical compositions including the foregoing are provided.

### DETAILED DESCRIPTION

The compositions described herein are drawn to targeting toxic PPP2R5D alleles thereby reducing production of mutant PPP2R5D for treatment of Jordan's Syndrome. Effective inhibition of mutant PPP2R5D by the RNAi oligonucleotides described herein results in a reduction in mutant PPP2R5D expression levels without interfering with non-mutant wild type PPP2R5D. Information relating to genomic PPP2R5D including sequence information is publicly available under Gene ID. 5528 (https://www.ncbi.nlm.nih.gov/gene/5528), protein phosphatase 2 regulatory subunit B′delta [Homo sapiens (human)]; and NCBI Reference Sequence: NG_050636.1.

RNAi(s) described herein are based on protein phosphatase 2 regulatory subunit B′delta (PPP2R5D), mRNA transcript variant 1, GenBank Accession NM_006245.4 (SEQ ID NO: 136); Homo sapiens, protein phosphatase 2 regulatory subunit B′delta (PPP2R5D), transcript variant 1, mRNA, NCBI Reference Sequence: NM_006245.4. PPP2R5D has proteins that correspond to UniProtKB identifier Q14738. As used herein, “mutant PPP2R5D” includes any PPP2R5D variant containing one or more of four individual PPP2R5D missense mutations (E197K, E198K, E200K and E420K). Certain PPP2R5D variants are described as the 10 transcripts referred to at PPP2R5D ENSG00000112640 (http://useast.ensembl.org/Homo_sapiens/Gene/Summary?g=ENSG00000112640; r=6:42984553-43012342).

The RNAi oligonucleotides described herein are siRNAs, shRNAs or ASOs. RNAi(s) herein may also be referred to herein as short interfering nucleic acids (siNAs). In embodiments, specific RNAi sequences (which can be delivered either as siRNA duplexes or ASOs or transcribed as shRNAs from plasmid DNA) target one or more of four individual PPP2R5D missense mutations (E197K (c.589G>A), E198K (c.592G>A), E200K (c.598G>A) and E420K (c.1258G>A)) (the “PPP2R5D missense mutations”) causative of Jordan Syndrome. Expression vectors encoding the shRNA sequences are provided. In embodiments, the expression vector is a lentiviral vector. In embodiments, the expression vector is an adeno-associated virus (AAV). In embodiments, siRNA is delivered by nanoparticulate vehicles or by polymeric vehicles.

In embodiments, provided herein are double stranded RNA molecules incorporating an antisense strand and a sense strand, wherein the nucleotide sequence of the antisense strand is complementary to a region of the nucleotide sequence of human mutant PPP2R5D. In embodiments, provided herein are double stranded RNA molecules incorporating an antisense strand and a sense strand, wherein the nucleotide sequence of the antisense strand is complementary to a region of the nucleotide sequence of human mutant PPP2R5D, based on transcript variant 1, mRNA (SEQ ID NO: 136). In embodiments, the region of the nucleotide sequence of human PPP2R5D contains one or more of the four individual PPP2R5D missense mutations.

Without wishing to be bound to a particular theory, the RNAi(s) herein may inhibit mutant PPP2R5D by: (1) cutting the RNA transcript encoded by mutant PPP2R5D having one or more of the PPP2R5D missense mutations; (2) reducing steady-state levels (i.e., baseline levels at homeostasis) of the RNA transcript encoded by mutant PPP2R5D having one or more of the PPP2R5D missense mutations; and/or (3) terminating transcription of mutant PPP2R5D having one or more of the PPP2R5D missense mutations.

siRNA molecules can consist of a characteristic 19+2mer structure (that is, a duplex of two 21-nucleotide RNA molecules with 19 complementary bases and terminal 2-nucleotide 3′ overhangs, such as dTdT on the 3′ end). One of the strands of the siRNA (the guide or antisense strand) is complementary to a target transcript, whereas the other strand is designated the passenger or sense strand. siRNAs act to guide the Argonaute 2 protein (AGO2), as part of the RNA-induced silencing complex (RISC), to complementary target transcripts. Complementarity between the siRNA and the target transcript results in cleavage of the target opposite position of the guide strand, catalyzed by AGO2 leading to gene silencing.

In embodiments, the siRNA sense strand is any of SEQ ID NOs: 1-29. In embodiments, the siRNA antisense strand is any of SEQ ID NOs: 30-58.

Non-complementary nucleobases between an antisense siRNA strand and a PPP2R5D nucleotide sequence may be tolerated provided that the antisense siRNA remains able to specifically hybridize to a PPP2R5D nucleotide sequence.

In embodiments, the siRNA may include a nucleotide sequence at least 85% complementary to, and of equal length as, any of SEQ ID NOs: 1-29. In embodiments, the siRNA may include a nucleotide sequence at least 90% complementary to, and of equal length as, any of SEQ ID NOs: 1-29. In embodiments, the siRNA may include a nucleotide at least 95% complementary to, and of equal length as, any of SEQ ID NOs: 1-29. In embodiments, the siRNA may encompass a nucleotide sequence 100% complementary to, and of equal length as, any of SEQ ID NOs: 1-29, in this case SEQ ID NOs: 30-58. A percent complementarity is used herein in the conventional sense to refer to base pairing between adenine and thymine, adenine and uracil (RNA), and guanine and cytosine.

In embodiments, the siRNA provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a PPP2R5D RNA nucleotide sequence. Percent complementarity of a siRNA with a PPP2R5D nucleotide sequence can be determined using routine methods.

For example, a siRNA antisense strand in which 18 of 20 nucleobases are complementary to a PPP2R5D region and would therefore specifically hybridize, would represent 90 percent complementarity. In this example, the remaining non-complementary nucleobases may be clustered or interspersed with complementary nucleobases and need not be contiguous to each other or to complementary nucleobases. As such, a siRNA which is 18 nucleobases in length having four non-complementary nucleobases which are flanked by two regions of complete complementarity with the target nucleotide sequence would have 77.8% overall complementarity with the target nucleotide sequence and would thus fall within the subject matter disclosed herein. Percent complementarity of a siRNA with a region of a PPP2R5D nucleotide sequence can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In embodiments, the antisense strand is a shortened or truncated modified oligonucleotide. The shortened or truncated modified oligonucleotide can have a single nucleoside deleted from the 5′ end (5′ truncation), the central portion or alternatively from the 3′ end (3′ truncation). A shortened or truncated oligonucleotide can have one or more nucleosides deleted from the 5′ end, one or more nucleosides deleted from the central portion or alternatively can have one or more nucleosides deleted from the 3′ end. Alternatively, the deleted nucleosides can be dispersed throughout the modified oligonucleotide, for example, in an antisense strand having one or more nucleoside deleted from the 5′ end, one or more nucleosides deleted from the central portion and/or one or more nucleoside deleted from the 3′ end.

In embodiments, siRNAs can include, without limitation, modified siRNAs, including siRNAs with enhanced stability in vivo. Modified siRNAs include molecules containing nucleotide analogues, including those molecules having additions, deletions, and/or substitutions in the nucleobase, sugar, or backbone; and molecules that are cross-linked or otherwise chemically modified. The modified nucleotide(s) may be within portions of the siRNA molecule, or throughout it. For instance, the siRNA molecule may be modified, or contain modified nucleic acids in regions at its 5′ end, its 3′ end, or both, and/or within the guide strand, passenger strand, or both, and/or within nucleotides that overhang the 5′ end, the 3′ end, or both. In embodiments, nucleic acids can be chemically modified at the backbone, nucleobase, ribose sugar and 2′-ribose substitutions modifications of RNA by, e.g., cEt, constrained ethyl bridged nucleic acid; ENA, ethylene-bridged nucleic acid; 2′-F, 2′-fluoro; LNA, locked nucleic acid; 2′-MOE, 2′-O-methoxyethyl; 2′-OMe, 2′-O-methyl; PMO, phosphorodiamidate morpholino oligonucleotide; PNA, peptide nucleic acid; phosphodiester bonds between the nucleotides could be replaced with phosphorothioate linkage, PS, phosphorothioate; tcDNA, tricyclo DNA.

shRNAs also involve RISC. Once a vector carrying the genomic material for the shRNA is integrated into the host genome, the shRNA genomic material is transcribed in the host into pri-microRNA. The pri-microRNA is processed by a ribonuclease, such as Drosha, into pre-shRNA and exported from the nucleus. The pre-shRNA is processed by an endoribonuclease such as Dicer to form siRNA. The siRNA is loaded into the RISC where the sense strand is degraded and the antisense strand acts as a guide that directs RISC to the complementary sequence in the mRNA. RISC cleaves the mRNA when the sequence has perfect complementary and represses translation of the mRNA when the sequence has imperfect complementary. Thus, the shRNA decreases or eliminates expression of the RNA transcript encoded by mutant PPP2R5D having one or more of the PPP2R5D missense mutations.

As used herein, a “short hairpin RNA (shRNA)” includes a conventional stem-loop shRNA, which forms a precursor microRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, a conventional shRNA forms a primary miRNA (pri-miRNA) or a structure very similar to a natural pri-miRNA. The pri-miRNA is subsequently processed by Drosha and its cofactors into pre-shRNA. Therefore, the term “shRNA” includes pri-miRNA (shRNA-mir) molecules and pre-shRNA molecules.

A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). It is known in the art that the loop portion is at least 4 nucleotides long, 6 nucleotides long, 8 nucleotides long, or more. The terms “hairpin” and “fold-back” structures are also used herein to refer to stem-loop structures. Such structures are well known in the art and the term is used consistently with its known meaning in the art. For example, DNA loop portions CTCGAG (SEQ ID NO: 59), TCAAGAG (SEQ ID NO: 60), TTCG (SEQ ID NO: 61), and GAAGCTTG (SEQ ID NO: 62) or RNA loop portions CUCGAG (SEQ ID NO: 63), UCAAGAG (SEQ ID NO: 64), UUCG (SEQ ID NO: 65), and GAAGCUUG (SEQ ID NO: 66) are suitable stem-loop structures. As is known in the art, the secondary structure does not require exact base-pairing. Thus, the stem can include one or more base mismatches or bulges. Alternatively, the base-pairing can be exact, i.e., not include any mismatches.

In embodiments, a shRNA sequence is provided which includes a first portion, a second portion and a third portion, the first portion comprising any of SEQ ID NOs: 1-29, the second portion comprising any of SEQ ID Nos: 59-66, and the third portion comprising respective nucleotide sequences complementary to those in SEQ ID NOs: 1-29, i.e., SEQ ID NOs: 30-58.

In embodiments, shRNAs can include, without limitation, modified shRNAs, including shRNAs with enhanced stability in vivo. Modified shRNAs include molecules containing nucleotide analogues, including those molecules having additions, deletions, and/or substitutions in the nucleobase, sugar, or backbone; and molecules that are cross-linked or otherwise chemically modified as discussed above. The modified nucleotide(s) may be within portions of the shRNA molecule, or throughout it. For instance, the shRNA molecule may be modified, or contain modified nucleic acids in regions at its 5′ end, its 3′ end, or both, and/or within the guide strand, passenger strand, or both, and/or within nucleotides that overhang the 5′ end, the 3′ end, or both.

In embodiments, polynucleotides encoding shRNA oligonucleotide sequences targeting individual PPP2R5D missense mutations are provided that result in decreased expression of mutant PPP2R5D. The polynucleotide may be a DNA polynucleotide suitable for cloning into an appropriate vector (e.g., a plasmid) for culturing and subsequent production of viruses or viral particles. In turn, viruses or viral particles may contain the DNA polynucleotide with the nucleotide coding sequence in a form suitable for infection. Thus, the polynucleotide may be a DNA sequence cloned into a plasmid for virus or viral particle production or encapsulated in a virus or viral particle. As retroviruses carry nucleotide coding sequences in the form of RNA polynucleotides, retroviral particles (e.g., lentivirus) may contain a shRNA that includes a nucleotide portion, a second portion and a third portion as described above.

In embodiments, polynucleotides encoding shRNA oligonucleotide sequences targeting individual PPP2R5D missense mutations encode a first portion, a second portion and a third portion, the first portion comprising any of SEQ ID NOs: 1-29, the second portion comprising any of SEQ ID Nos: 59-66, and the third portion comprising respective nucleotide sequences complementary to those in SEQ ID NOs: 1-29, i.e., SEQ ID NOs: 30-58.

“Reduce expression”, “decease expression” or “inhibit expression” refers to a reduction or blockade of the expression or activity of mutant PPP2R5D and does not necessarily indicate a total elimination of expression or activity. Mechanisms for reduced expression of the target include hybridization of an operative RNA polynucleotide with a target sequence or sequences transcribed from a sequence or sequences within the larger genomic mutant PPP2R5D sequence, wherein the outcome or effect of the hybridization is either target degradation or target occupancy with concomitant stalling of the cellular machinery involving, for example, transcription or splicing.

As discussed below, ASOs are provided that target common SNPs associated with multiple PPP2R5D missense mutations thereby reducing mutant PPP2R5D expression. Classic single stranded ASOs primarily act in the nucleus by selectively cleaving pre-mRNAs having complementary sites via an RNase H dependent mechanism. Although ASOs can also act by translation arrest via steric hinderance, in embodiments, they are used as ‘gapmers’, having a central region that supports RNase H activity flanked by chemically modified ends that increase affinity and reduce susceptibility to nucleases. The endogenous RNase H enzyme RNASEH1 recognizes RNA-DNA heteroduplex substrates that are formed when DNA-based oligonucleotides bind to their cognate mRNA transcripts and catalyzes the degradation of RNA. Cleavage at the site of ASO binding results in destruction of the target RNA, thereby silencing target gene expression.

Gapmer ASOs, consisting of a DNA-based internal ‘gap’ and RNA-like flanking regions (optionally consisting of 2′-O-methyl (2′-OMe) or locked nucleic acid (LNA) modified bases) bind to target transcripts with high affinity. The resulting RNA-DNA duplex acts as a substrate for RNASEH1, leading to the degradation of the target transcript.

One skilled in the art will understand that complementarity to the PPP2R5D mRNA can be established using canonical nucleotides comprising ribose, phosphate and one of the bases adenine, guanine, cytosine, and uracil linked with the phosphodiester linkages typifying naturally occurring nucleic acids. In embodiments, as mentioned above, nucleic acids can be chemically modified at the backbone, nucleobase, ribose sugar and 2′-ribose substitutions modifications of RNA by, e.g., cEt, constrained ethyl bridged nucleic acid; ENA, ethylene-bridged nucleic acid; 2′-F, 2′-fluoro; LNA, locked nucleic acid; 2′-MOE, 2′-O-methoxyethyl; 2′-OMe, 2′-O-methyl; PMO, phosphorodiamidate morpholino oligonucleotide; PNA, peptide nucleic acid; phosphodiester bonds between the nucleotides could be replaced with phosphorothioate linkage, PS, phosphorothioate; tcDNA, tricyclo DNA.

As used herein, the term “nucleic acid” refers to molecules composed of monomeric nucleotides. Examples of nucleic acids include ribonucleic acids (RNA), deoxyribonucleic acids (DNA), single-stranded nucleic acids, double-stranded nucleic acids, small interfering ribonucleic acids (siRNA), and short hairpin RNAs (shRNAs), microRNAs, pri-microRNAs, pre-shRNAs and ASOs. “Nucleic acid” includes oligonucleotides and polynucleotides. “Nucleotide” means a nucleoside having a phosphate group covalently linked to the sugar portion of the nucleoside. “Oligonucleotide” or “polynucleotide” may be used interchangeably herein and mean a polymer of linked nucleotides each of which can be modified or unmodified, independent one from another.

Table 1 lists novel siRNA/shRNA oligonucleotide sequences targeting individual PPP2R5D missense mutations.

TABLE 1
siRNA/shRNA sequences targeting individual PPP2R5D missense mutations

| | | | |
|---|---|---|---|
| SEQUENCE ID | Oligo Sense Sequence | SEQUENCE ID | Oligo Antisense Sequence |

E197K

| | | | |
|---|---|---|---|
| SEQ ID NO: 1 | CAAAGGAAGAUGAGCCCAC | SEQ ID NO: 30 | GUGGGCUCAUCUUCCUUUG |
| SEQ ID NO: 2 | AAAGGAAGAUGAGCCCACC | SEQ ID NO: 31 | GGUGGGCUCAUCUUCCUUU |
| SEQ ID NO: 3 | CCAAAGGAAGAUGAGCCCA | SEQ ID NO: 32 | UGGGCUCAUCUUCCUUUGG |
| SEQ ID NO: 4 | ACCCAAAGGAAGAUGAGCC | SEQ ID NO: 33 | GGCUCAUCUUCCUUUGGGU |
| SEQ ID NO: 5 | CCCAAAGGAAGAUGAGCCC | SEQ ID NO: 34 | GGGCUCAUCUUCCUUUGGG |
| SEQ ID NO: 6 | CAAAGGAAGAUGAGCCCACCC | SEQ ID NO: 35 | GUGGGCUCAUCUUCCUUUGGG |
| SEQ ID NO: 7 | AAAGGAAGAUGAGCCCACCCU | SEQ ID NO: 36 | GGUGGGCUCAUCUUCCUUUGG |

E198K

| | | | |
|---|---|---|---|
| SEQ ID NO: 8 | CCAGAGAAAGAUGAGCCCA | SEQ ID NO: 37 | UGGGCUCAUCUUUCUCUGG |
| SEQ ID NO: 9 | CAGAGAAAGAUGAGCCCAC | SEQ ID NO: 38 | GUGGGCUCAUCUUUCUCUG |
| SEQ ID NO: 10 | ACCCAGAGAAAGAUGAGCC | SEQ ID NO: 39 | GGCUCAUCUUUCUCUGGGU |
| SEQ ID NO: 11 | CCCAGAGAAAGAUGAGCCC | SEQ ID NO: 40 | GGGCUCAUCUUUCUCUGGG |
| SEQ ID NO: 12 | AGAGAAAGAUGAGCCCACCCU | SEQ ID NO: 41 | GGUGGGCUCAUCUUUCUCUGG |
| SEQ ID NO: 13 | GAGAAAGAUGAGCCCACCCUG | SEQ ID NO: 42 | GGGUGGGCUCAUCUUUCUCUG |

E420K

| | | | |
|---|---|---|---|
| SEQ ID NO: 14 | CCCCAUUUCCAGGUGGCAA | SEQ ID NO: 43 | UUGCCACCUGGAAAUGGGG |
| SEQ ID NO: 15 | CCCAUUUCCAGGUGGCAAA | SEQ ID NO: 44 | UUUGCCACCUGGAAAUGGG |
| SEQ ID NO: 16 | CAUUUCCAGGUGGCAAAGC | SEQ ID NO: 45 | GCUUUGCCACCUGGAAAUG |
| SEQ ID NO: 17 | CCAUUUCCAGGUGGCAAAG | SEQ ID NO: 46 | CUUUGCCACCUGGAAAUGG |
| SEQ ID NO: 18 | CCCCAUUUCCAGGUGGCAAAG | SEQ ID NO: 47 | UUGCCACCUGGAAAUGGGGGC |
| SEQ ID NO: 19 | UUUCCAGGUGGCAAAGCGUGC | SEQ ID NO: 48 | ACGCUUUGCCACCUGGAAAUG |

E200K

| | | | |
|---|---|---|---|
| SEQ ID NO: 20 | UUUGACCCAGAGGAAGAUA | SEQ ID NO: 49 | UAUCUUCCUCUGGGUCAAA |
| SEQ ID NO: 21 | UUGACCCAGAGGAAGAUAA | SEQ ID NO: 50 | UUAUCUUCCUCUGGGUCAA |
| SEQ ID NO: 22 | UGACCCAGAGGAAGAUAAG | SEQ ID NO: 51 | CUUAUCUUCCUCUGGGUCA |
| SEQ ID NO: 23 | UGACCCAGAGGAAGAUAAGCC | SEQ ID NO: 52 | CUUAUCUUCCUCUGGGUCAAA |
| SEQ ID NO: 24 | GACCCAGAGGAAGAUAAGCCC | SEQ ID NO: 53 | GCUUAUCUUCCUCUGGGUCAA |
| SEQ ID NO: 25 | ACCCAGAGGAAGAUAAGCCCA | SEQ ID NO: 54 | GGCUUAUCUUCCUCUGGGUCA |
| SEQ ID NO: 26 | GAGGAAGAUAAGCCCACCC | SEQ ID NO: 55 | GGGUGGGCUUAUCUUCCUC |
| SEQ ID NO: 27 | GAGGAAGAUAAGCCCACCCUG | SEQ ID NO: 56 | GGGUGGGCUUAUCUUCCUCUG |
| SEQ ID NO: 28 | AAGAUAAGCCCACCCUGGAAG | SEQ ID NO: 57 | UCCAGGGUGGGCUUAUCUUCC |
| SEQ ID NO: 29 | GCCCACCCUGGAAGCUGCUUG | SEQ ID NO: 58 | AGCAGCUUCCAGGGUGGGCUU |

Table 2 lists novel polynucleotides encoding siRNA/shRNA oligonucleotide sequences targeting individual PPP2R5D missense mutations.

TABLE 2
Polynucleotides encoding siRNA/shRNA oligonucleotide sequences
targeting individual PPP2R5D missense mutations

| | | | |
|---|---|---|---|
| Sequence ID | Sense Sequence | Sequence ID | Antisense Sequence |

E197K

| | | | |
|---|---|---|---|
| SEQ ID NO: 67 | CAAAGGAAGATGAGCCCAC | SEQ ID NO: 96 | GTGGGCTCATCTTCCTTTG |
| SEQ ID NO: 68 | AAAGGAAGATGAGCCCACC | SEQ ID NO: 97 | GGTGGGCTCATCTTCCTTT |
| SEQ ID NO: 69 | CCAAAGGAAGATGAGCCCA | SEQ ID NO: 98 | TGGGCTCATCTTCCTTTGG |
| SEQ ID NO: 70 | ACCCAAAGGAAGATGAGCC | SEQ ID NO: 99 | GGCTCATCTTCCTTTGGGT |
| SEQ ID NO: 71 | CCCAAAGGAAGATGAGCCC | SEQ ID NO: 100 | GGGCTCATCTTCCTTTGGG |
| SEQ ID NO: 72 | CAAAGGAAGATGAGCCCACCC | SEQ ID NO: 101 | GTGGGCTCATCTTCCTTTGGG |

E198K

| | | | |
|---|---|---|---|
| SEQ ID NO: 73 | AAAGGAAGATGAGCCCACCCT | SEQ ID NO: 102 | GGTGGGCTCATCTTCCTTTGG |
| SEQ ID NO: 74 | CCAGAGAAAGATGAGCCCA | SEQ ID NO: 103 | TGGGCTCATCTTTCTCTGG |
| SEQ ID NO: 75 | CAGAGAAAGATGAGCCCAC | SEQ ID NO: 104 | GTGGGCTCATCTTTCTCTG |
| SEQ ID NO: 76 | ACCCAGAGAAAGATGAGCC | SEQ ID NO: 105 | GGCTCATCTTTCTCTGGGT |
| SEQ ID NO: 77 | CCCAGAGAAAGATGAGCCC | SEQ ID NO: 106 | GGGCTCATCTTTCTCTGGG |
| SEQ ID NO: 78 | AGAGAAAGATGAGCCCACCCT | SEQ ID NO: 107 | GGTGGGCTCATCTTTCTCTGG |
| SEQ ID NO: 79 | GAGAAAGATGAGCCCACCCTG | SEQ ID NO: 108 | GGGTGGGCTCATCTTTCTCTG |

E420K

| | | | |
|---|---|---|---|
| SEQ ID NO: 80 | CCCCATTTCCAGGTGGCAA | SEQ ID NO: 109 | TTGCCACCTGGAAATGGGG |
| SEQ ID NO: 81 | CCCATTTCCAGGTGGCAAA | SEQ ID NO: 110 | TTTGCCACCTGGAAATGGG |
| SEQ ID NO: 82 | CATTTCCAGGTGGCAAAGC | SEQ ID NO: 111 | GCTTTGCCACCTGGAAATG |
| SEQ ID NO: 83 | CCATTTCCAGGTGGCAAAG | SEQ ID NO: 112 | CTTTGCCACCTGGAAATGG |
| SEQ ID NO: 84 | CCCCATTTCCAGGTGGCAAAG | SEQ ID NO: 113 | TTGCCACCTGGAAATGGGGGC |
| SEQ ID NO: 85 | TTTCCAGGTGGCAAAGCGTGC | SEQ ID NO: 114 | ACGCTTTGCCACCTGGAAATG |

E200K

| | | | |
|---|---|---|---|
| SEQ ID NO: 86 | TTTGACCCAGAGGAAGATA | SEQ ID NO: 115 | GGACTTGCCGGCACCCATCTG |
| SEQ ID NO: 87 | TTGACCCAGAGGAAGATAA | SEQ ID NO: 116 | CTTGCCGGCACCCATCTGCCC |
| SEQ ID NO: 88 | TGACCCAGAGGAAGATAAG | SEQ ID NO: 117 | ATCTGCCCATAGGCGAAGATG |
| SEQ ID NO: 89 | TGACCCAGAGGAAGATAAGCC | SEQ ID NO: 118 | CTTGCCGGCACCCATCTGCC |
| SEQ ID NO: 90 | GACCCAGAGGAAGATAAGCCC | SEQ ID NO: 119 | GACTTGCCGGCACCCATCTGC |
| SEQ ID NO: 91 | ACCCAGAGGAAGATAAGCCCA | SEQ ID NO: 120 | CGCTTGCTCCCAGCCAGGT |
| SEQ ID NO: 92 | GAGGAAGATAAGCCCACCC | SEQ ID NO: 121 | CCGCTTGCTCCCAGCCAGG |
| SEQ ID NO: 93 | GAGGAAGATAAGCCCACCCTG | SEQ ID NO: 122 | CCCGCTTGCTCCCAGCCAG |
| SEQ ID NO: 94 | AAGATAAGCCCACCCTGGAAG | SEQ ID NO: 123 | TCAGCCCGCTTGCTCCCAG |
| SEQ ID NO: 95 | GCCCACCCTGGAAGCTGCTTG | SEQ ID NO: 124 | GAGTCAGCCCGCTTGCTCC |

Shown in Table 3 are 20nt MOE Gapmer ASOs targeting E198K PPP2R5D missense mutation. In embodiments. ASOs could be truncated. i.e., 19nt, 18nt, 17nt, 16nt, or 15nt. They could be of the 5-10-5 format or 5-10-4, 4-10-4, 4-10-3, 3-10-3, 3-10-2, 5-9-6, 5-9-5, 4-9-5, 4-9-4, 3-9-4, 3-9-3, 6-8-6, 6-8-5, 5-8-5, 5-8-4, 4-8-4, or 4-8-3.

TABLE 3
Allele-specific antisense oligonucleotide
(ASO) designs targeting E198K PPP2R5D
missense mutation

| Sequence ID | ASO sequence |
|---|---|
| SEQ ID NO: 125 | UCUUUCUCUGGGUCAAACUC |
| SEQ ID NO: 126 | AUCUUUCUCUGGGUCAAACU |
| SEQ ID NO: 127 | CAUCUUUCUCUGGGUCAAAC |
| SEQ ID NO: 128 | UCAUCUUUCUCUGGGUCAAA |
| SEQ ID NO: 129 | CUCAUCUUUCUCUGGGUCAA |
| SEQ ID NO: 130 | GCUCAUCUUUCUCUGGGUCA |
| SEQ ID NO: 131 | GGCUCAUCUUUCUCUGGGUC |
| SEQ ID NO: 132 | GGGCUCAUCUUUCUCUGGGU |
| SEQ ID NO: 133 | UGGGCUCAUCUUUCUCUGGG |
| SEQ ID NO: 134 | GUGGGCUCAUCUUUCUCUGG |
| SEQ ID NO: 135 | GGUGGGCUCAUCUUUCUCUG |

The oligonucleotides described herein may be conveniently and routinely made by known techniques, e.g., solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the substituted sugars, phosphorothioates and alkylated derivatives.

In embodiments, lipid nanoparticles can be used to deliver the oligonucleotides. In embodiments, anionic oligonucleotides are complexed with cationic lipids thus forming lipid nanoparticles (LNPs). LNPs for in vivo use can be 100-200 nm in size and include a surface coating of a neutral polymer such as PEG to minimize protein binding and uptake by RES cells. The action of LNPs involves initial uptake by endocytosis. Once in endosomes, the cationic lipids of the LNP interact with anionic membrane lipids to disrupt membrane structure. This leads both to increased membrane permeability and to dissolution of the LNP and is the basis for conveying the oligonucleotide to the cytosol.

In embodiments, stable-nucleic-acid lipid particles (SNALPs) can be utilized for siRNA delivery. Optimized cationic lipids may be obtained by (i) altering the pKa so that the lipids are almost uncharged in the circulation but become charged in the low pH endosome and (ii) using linkages that are readily biodegradable. This results in dramatic improvement in effectiveness, allowing siRNA or shRNA doses as low as 0.005 mg/kg to achieve significant silencing of targets, accompanied by low toxicity.

In embodiments, polymeric nanocarriers can be utilized for oligonucleotide delivery. Biomedically compatible polymers such as poly lactide, polyglycolide or poly(lactic-co-glycolic acid) (PLGA) can be utilized to form solid nanoparticles through oil-in-water emulsion techniques. Since PLGA is anionic, positive side chains in the polymer may be incorporated or the anionic oligonucleotide can be complexed with a positively charged moiety such as polyethylene imine (PEI).

In embodiments, micelle polymeric nanocarriers may be used for oligonucleotide delivery. These may be formed by self-assembly of amphiphilic polymers in a water environment. For example, a polymeric micelle may be formed from a tri-block polymer including a hydrophobic portion to drive self-assembly, a cationic portion to bind the oligonucleotide and PEG or other neutral polymer to provide a protective coating. In embodiments, another polymeric nanocarrier is a nanohydrogel. These nanoparticles have an open, water-filled polymer lattice that easily incorporate bio-macromolecules such as polypeptides and oligonucleotides, whose release kinetics are controlled by the degree of cross linking of the lattice.

In embodiments, oligonucleotides described herein may be incorporated into plasmids, viral vectors, or viral particles.

A “vector” is a replicon, such as a plasmid, phage, or cosmid, into which a DNA segment or an RNA segment may be inserted so as to bring about the replication of the inserted segment. Generally, a vector is capable of replication when associated with the proper control elements. Suitable vector backbones include, for example, those routinely used in the art such as plasmids, plasmids that contain a viral genome, viruses, or artificial chromosomes. The term “vector” includes cloning and expression vectors, as well as viral vectors and integrating vectors.

As will be evident to one of skill in the art, the term “viral vector” is widely used to refer to a nucleic acid molecule (e.g., a transfer plasmid) that includes viral nucleic acid elements that typically facilitate transfer of the nucleic acid molecule to a cell or to a viral particle that mediates nucleic acid sequence transfer and/or integration of the nucleic acid sequence into the genome of a cell.

Viral vectors contain structural and/or functional genetic elements that are primarily derived from a virus. The viral vector is desirably non-toxic, non-immunogenic, easy to produce, and efficient in protecting and delivering DNA or RNA into the target cells. According to the compositions and methods described herein a viral vector may contain the DNA that encodes one or more of the siRNAs, shRNAs and dsRNAs, described herein. In embodiments, the viral vector is a lentiviral vector or an adeno-associated viral (AAV) vector.

As used herein, the term “lentivirus” refers to a group (or genus) of complex retroviruses. Illustrative lentiviruses include but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). As used herein, the term “lentivirus” includes lentivirus particles. Lentivirus will transduce dividing cells and postmitotic cells.

The term “lentiviral vector” refers to a viral vector (e.g., viral plasmid) containing structural and functional genetic elements, or portions thereof, including long terminal repeats (LTRs) that are primarily derived from a lentivirus. A lentiviral vector is a hybrid vector (e.g., in the form of a transfer plasmid) having retroviral, e.g., lentiviral, sequences for reverse transcription, replication, integration and/or packaging of nucleic acid sequences (e.g., coding sequences). The term “retroviral vector” refers to a viral vector (e.g., transfer plasmid) containing structural and functional genetic elements, or portions thereof, that are primarily derived from a retrovirus.

Adenoviral vectors are designed to be administered directly to a living subject. Unlike retroviral vectors, most of the adenoviral vector genomes do not integrate into the chromosome of the host cell. Instead, genes introduced into cells using adenoviral vectors are maintained in the nucleus as an extrachromosomal element (episome) that persists for an extended period of time. Adenoviral vectors will transduce dividing and non-dividing cells in many different tissues in vivo including airway epithelial cells, endothelial cells, hepatocytes, and various tumors.

The term “adeno-associated virus” (AAV) refers to a small ssDNA virus which infects humans and some other primate species, not known to cause disease, and causes only a very mild immune response. As used herein, the term “AAV” is meant to include AAV particles. AAV can infect both dividing and non-dividing cells and may incorporate its genome into that of the host cell. These features make AAV an attractive candidate for creating viral vectors for gene therapy, although the cloning capacity of the vector is relatively limited. In embodiments, the vector used is derived from adeno-associated virus (i.e., AAV vector). More than 30 naturally occurring serotypes of AAV are available. Many natural variants in the AAV capsid exist, allowing identification and use of an AAV with properties specifically suited for specific types of target cells. AAV viruses may be engineered by conventional molecular biology techniques, making it possible to optimize these particles for cell specific delivery of shRNA DNA sequences, for minimizing immunogenicity, for tuning stability and particle lifetime, for efficient degradation, for accurate delivery to the nucleus, etc.

An “expression vector” is a vector that includes a regulatory region. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.). An expression vector may be a viral expression vector derived from a particular virus.

The vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of pLK0.1 puro, SV40 and, plasmids such as RP4; phage DNAs, e.g., the numerous derivatives of phage 1, e.g., NM989, and other phage DNA, e.g., M13 and filamentous single stranded phage DNA, vectors useful in eukaryotic cells, such as vectors useful in insect or mammalian cells, vectors derived from combinations of plasmids and phage DNAs, such as plasmids that have been modified to employ phage DNA or other expression control sequences.

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically includes at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. Modulation of the expression of a coding sequence can be accomplished by appropriately selecting and positioning promoters and other regulatory regions relative to the coding sequence.

Vectors can also include other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available.

A “recombinant viral vector” refers to a viral vector including one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation).

In embodiments, the viral vector used herein can be used, e.g., at a concentration of at least 10⁵ viral genomes per cell.

The selection of appropriate promoters can readily be accomplished. Examples of suitable promoters include RNA polymerase II or III promoters. For example, candidate shRNA sequences may be expressed under control of RNA polymerase III promoters U6 or H1, or neuron-specific RNA polymerase II promoters including neuron-specific enolase (NSE), synapsin I (Syn), or the Ca2+/CaM-activated protein kinase II alpha (CaMKIIalpha).

Other suitable promoters which may be used for gene expression include, but are not limited to, the 763-base-pair cytomegalovirus (CMV) promoter, the Rous sarcoma virus (RSV) (Davis, et al., Hum Gene Ther 4:151 (1993)), the SV40 early promoter region, the herpes thymidine kinase promoter, the regulatory sequences of the metallothionein (MMT) gene, PGK (phosphoglycerol kinase) promoter, alkaline phosphatase promoter; and the animal transcriptional control regions, which exhibit tissue specificity and have been utilized in transgenic animals: myelin basic protein gene control region which is active in oligodendrocyte cells in the brain, and gonadotropic releasing hormone gene control region which is active in the hypothalamus. Certain proteins can be expressed using their native promoter. Other elements that can enhance expression can also be included such as an enhancer or a system that results in high levels of expression such as a tat gene and tar element. The assembly or cassette can then be inserted into a vector, e.g., a plasmid vector such as, pLK0.1, pUC19, pUC118, pBR322, or other known plasmid vectors. See, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely affect the metabolism of the organism being treated.

Coding sequences for RNAi(s) herein can be cloned into viral vectors using any suitable genetic engineering technique well known in the art, including, without limitation, the standard techniques of PCR, polynucleotide synthesis, restriction endonuclease digestion, ligation, transformation, plasmid purification, and DNA sequencing, as described in Sambrook et al. (Molecular Cloning: A Laboratory Manual. Cold Spring Harbor Laboratory Press, N.Y. (1989)), Coffin et al. (Retroviruses. Cold Spring Harbor Laboratory Press, N.Y. (1997)) and “RNA Viruses: A Practical Approach” (Alan J. Cann, Ed., Oxford University Press, (2000)). In embodiments, the RNAi, e.g., shRNA DNA sequences contain flanking sequences on the 5′ and 3′ ends that are complementary with sequences on the plasmid and/or vector that is cut by a restriction endonuclease. As is well known in the art, the flanking sequences depend on the restriction endonucleases used during the restriction digest of the plasmid and/or vector. Thus, one of skill in the art can select the flanking sequences on the 5′ and 3′ ends of the RNAi DNA sequences accordingly. In embodiments, the target sites can be cloned into vectors by nucleic acid fusion and exchange technologies currently known in the art, including, Gateway, PCR in fusion, Cre-lox P, and Creator.

In embodiments, an expression vector includes a promoter and a polynucleotide including a first nucleotide sequence encoding a shRNA described herein. In embodiments, the promoter and the polynucleotide including the first nucleotide sequence are operably linked. In embodiments, the promoter is a U6 promoter. In embodiments, the first nucleotide sequence included in the expression vector may be polynucleotides encoding SEQ ID NOs: 1-29. In embodiments, the first nucleotide sequence included in the expression vector may include any of SEQ ID NOs: 67-95. In embodiments, an expression vector includes a polynucleotide including a second nucleotide sequence encoding a shRNA described herein. In embodiments, the second nucleotide sequence included in the expression vector may be polynucleotides encoding SEQ ID NOs: 59-66. In embodiments, an expression vector includes a promoter and a polynucleotide including a third nucleotide sequence encoding a shRNA described herein. In embodiments, the third nucleotide sequence included in the expression vector may be polynucleotides encoding SEQ ID NOs: 30-58. In embodiments, the third nucleotide sequence included in the expression vector may include any of SEQ ID NOs: 96-124.

As discussed below, recombinant viral vectors are transfected into packaging cells or cell lines, along with elements required for the packaging of recombinant viral particles. Recombinant viral particles collected from transfected cell supernatant are used to infect target cells or organisms for the expression of shRNAs. The transduced cells or organisms are used for transient expression or selected for stable expression.

In embodiments, viral particles are used to deliver coding nucleotide sequences for the siRNA or shRNAs. The terms virus and viral particles are used interchangeably herein. Viral particles will typically include various viral components and sometimes also host cell components in addition to nucleic acid(s). Nucleic acid sequences may be packaged into a viral particle that is capable of delivering the siRNA or shRNA nucleic acid sequences into the target cells in the patient in need.

The viral particles may be produced by (a) introducing a viral expression vector into a suitable cell line; (b) culturing the cell line under suitable conditions so as to allow the production of the viral particle; (c) recovering the produced viral particle; and (d) optionally purifying the recovered infectious viral particle.

An expression vector containing the nucleotide sequence encoding one or more of the siRNA or shRNA herein may be introduced into an appropriate cell line for propagation or expression using well-known techniques readily available to the person of ordinary skill in the art. These include, but are not limited to, microinjection of minute amounts of DNA or RNA into the nucleus of a cell, CaPO₄-mediated transfection, DEAE-dextran-mediated transfection, electroporation, lipofection/liposome fusion, particle bombardment, gene guns, transduction, infection (e.g. with an infective viral particle), and other techniques such as those found in Sambrook, et al. (Molecular Cloning: A Laboratory Manual, Cold Spring Harbor Laboratory, Cold Spring Harbor Laboratory Press, Cold Spring Harbor, N.Y., 2001).

In embodiments, where an expression vector is defective, infectious particles can be produced in a complementation cell line or via the use of a helper virus, which supplies in trans the non-functional viral genes. For example, suitable cell lines for complementing adenoviral vectors include the 293 cells as well as the PER-C6 cells commonly used to complement the E1 function. The infectious viral particles may be recovered from the culture supernatant but also from the cells after lysis and optionally are further purified according to standard techniques such as chromatography, ultracentrifugation in a cesium chloride gradient and the like.

In embodiments, provided herein are host cells which include the nucleic acid molecules, vectors, or infectious viral particles described herein. The term “host cell” should be understood broadly without any limitation concerning particular organization in tissue, organ, or isolated cells. Such cells may be of a unique type of cells or a group of different types of cells and encompass cultured cell lines, primary cells, and proliferative cells.

Host cells therefore include prokaryotic cells, lower eukaryotic cells such as yeast, and other eukaryotic cells such as insect cells, plant and higher eukaryotic cells, such as vertebrate cells and, with a special preference, mammalian (e.g., human or non-human) cells. Suitable mammalian cells include but are not limited to hematopoietic cells (totipotent, stem cells, leukocytes, lymphocytes, monocytes, macrophages, APC, dendritic cells, non-human cells and the like), pulmonary cells, tracheal cells, hepatic cells, epithelial cells, endothelial cells, muscle cells (e.g., skeletal muscle, cardiac muscle or smooth muscle) or fibroblasts. For example, host cells can include Escherichia coli, Bacillus, Listeria, Saccharomyces, BHK (baby hamster kidney) cells, MDCK cells (Madin-Darby canine kidney cell line), CRFK cells (Crandell feline kidney cell line), CV-1 cells (African monkey kidney cell line), COS (e.g., COS-7) cells, chinese hamster ovary (CHO) cells, mouse NIH/3T3 cells, HeLa cells and Vero cells. Host cells also encompass complementing cells capable of complementing at least one defective function of a replication-defective vector utilizable herein (e.g., a defective adenoviral vector) such as those cited above.

In embodiments, the host cell may be encapsulated. For example, transfected or infected eukaryotic host cells can be encapsulated with compounds which form a microporous membrane and said encapsulated cells may further be implanted in vivo. Capsules containing the cells of interest may be prepared employing hollow microporous membranes having a molecular weight cutoff appropriate to permit the free passage of proteins and nutrients between the capsule interior and exterior, while preventing the contact of transplanted cells with host cells.

Viral particles suitable for use herein include AAV particles and lentiviral particles. AAV particles carry the coding sequences for siRNAs or shRNAs herein in the form of DNA. Lentiviral particles, on the other hand, belong to the class of retroviruses and carry the coding sequences for siRNAs or shRNAs herein in the form of RNA.

Recombinantly engineered viral particles such as AAV particles, artificial AAV particles, self-complementary AAV particles, and lentiviral particles that contain the DNA (or RNA in the case of lentiviral particles) encoding the siRNAs or shRNAs targeting mutant PPP2R5D RNA or wild-type PPP2R5D RNA may be delivered to target cells to reduce expression of PPP2R5D. The use of AAVs is a common mode of delivery of DNA as it is relatively non-toxic, provides efficient gene transfer, and can be easily optimized for specific purposes. In embodiments, the selected AAV serotype has native neurotropisms. In embodiments, the AAV serotype can be AAV9 or AAV10.

A suitable recombinant AAV can be generated by culturing a host cell which contains a nucleotide sequence encoding an AAV serotype capsid protein, or fragment thereof, as defined herein; a functional rep gene; a minigene composed of, at a minimum, AAV inverted terminal repeats (ITRs) and a coding nucleotide sequence; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

Unless otherwise specified, the AAV inverted terminal repeats (ITRs), and other selected AAV components described herein, may be readily selected from among any AAV serotype, including, without limitation, AAV1, AAV2, AAV3, AAV5, AAV6, AAV7, AAV8, AAV9, AAV10, AAVRec3 or other known and unknown AAV serotypes. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

The minigene, rep sequences, cap sequences, and helper functions required for producing a rAAV herein may be delivered to the packaging host cell in the form of any genetic element which transfers the sequences carried thereon. The selected genetic element may be delivered by any suitable method. The methods used to construct embodiments herein are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation. See, e.g., K. Fisher et al, 1993 J. Viral., 70:520-532 and U.S. Pat. No. 5,478,745, among others. All citations herein are incorporated by reference herein.

Selection of these and other common vector and regulatory elements are conventional and many such sequences are available. See, e.g., Sambrook et al, and references cited therein at, for example, pages 3.18-3.26 and 16.17-16.27 and Ausubel et al., Current Protocols in Molecular Biology, John Wiley & Sons, New York, 1989). Of course, not all vectors and expression control sequences will function equally well to express all of the transgenes herein. However, one of skill in the art may make a selection among these, and other, expression control sequences.

The virus including the desired coding sequences for the siRNAs or shRNAs, can be formulated for administration to a patient or human in need by any means suitable for administration. Such formulation involves the use of a pharmaceutically and/or physiologically acceptable vehicle or carrier, particularly one suitable for administration to the brain, e.g., by subcranial or spinal injection. Further, more than one of the siRNAs, shRNAs or ASOs herein may be administered in a combination treatment. In a combination treatment, the different siRNAs, shRNAs or ASOs may be administered simultaneously, separately, sequentially, and in any order.

Pharmaceutical compositions containing the siRNAs, shRNAs or ASOs herein may include a carrier and/or diluent appropriate for its delivering by injection to a human or animal organism. Such carrier and/or diluent should be generally non-toxic at the dosage and concentration employed. It can be selected from those usually employed to formulate compositions for parental administration in either unit dosage or multi-dose form or for direct infusion by continuous or periodic infusion. In embodiments, it is isotonic, hypotonic or weakly hypertonic and has a relatively low ionic strength, such as provided by sugars, polyalcohols and isotonic saline solutions. Representative examples include sterile water, physiological saline (e.g., sodium chloride), bacteriostatic water, Ringer's solution, glucose or saccharose solutions, Hank's solution, and other aqueous physiologically balanced salt solutions (see for example the most current edition of Remington: The Science and Practice of Pharmacy, A. Gennaro, Lippincott, Williams & Wilkins). The pH of the composition is suitably adjusted and buffered in order to be appropriate for use in humans or animals, e.g., at a physiological or slightly basic pH (between about pH 8 to about pH 9, with a special preference for pH 8.5). Suitable buffers include phosphate buffer (e.g., PBS), bicarbonate buffer and/or Tris buffer. In embodiments, e.g., a composition is formulated in IM saccharose, 150 mM NaCl, 1 mM MgCl₂, 54 mg/l Tween 80, 10 mM Tris pH 8.5. In embodiments, e.g., a composition is formulated in 10 mg/ml mannitol, 1 mg/ml HSA, 20 mM Tris, pH 7.2, and 150 mM NaCl. These compositions are stable at −70° C. for at least six months.

Pharmaceutical compositions herein may be in various forms, e.g., in solid (e.g. powder, lyophilized form), or liquid (e.g. aqueous). In the case of solid compositions, methods of preparation are, e.g., vacuum drying and freeze-drying which yields a powder of the active agent plus any additional desired ingredient from a previously sterile-filtered solution thereof. Such solutions can, if desired, be stored in a sterile ampoule ready for reconstitution by the addition of sterile water for ready injection.

Nebulized or aerosolized formulations are also suitable. Methods of intranasal administration are well known in the art, including the administration of a droplet, spray, or dry powdered form of the composition into the nasopharynx of the individual to be treated from a pressured container or dispenser which contains a suitable propellant, e.g., a gas such as carbon dioxide, or a nebulizer. Enteric formulations such as gastroresistant capsules and granules for oral administration, suppositories for rectal or vaginal administration may also be suitable. For non-parental administration, the compositions can also include absorption enhancers which increase the pore size of the mucosal membrane. Such absorption enhancers include sodium deoxycholate, sodium glycocholate, dimethyl-beta-cyclodextrin, lauroyl-1-lysophosphatidylcholine and other substances having structural similarities to the phospholipid domains of the mucosal membrane.

The composition can also contain other pharmaceutically acceptable excipients for providing desirable pharmaceutical or pharmacodynamic properties, including for example modifying or maintaining the pH, osmolarity, viscosity, clarity, color, sterility, stability, rate of dissolution of the formulation, modifying or maintaining release or absorption into a human or animal organism. For example, polymers such as polyethylene glycol may be used to obtain desirable properties of solubility, stability, half-life and other pharmaceutically advantageous properties. Representative examples of stabilizing components include polysorbate 80, L-arginine, polyvinylpyrrolidone, trehalose, and combinations thereof. Other stabilizing components especially suitable in plasmid-based compositions include hyaluronidase, chloroquine, protic compounds such as propylene glycol, polyethylene glycol, glycerol, ethanol, 1-methyl L-2-pyrrolidone or derivatives thereof, aprotic compounds such as dimethylsulfoxide (DMSO), diethylsulfoxide, di-n-propylsulfoxide, dimethylsulfone, sulfolane, dimethyl-formamide, dimethylacetamide, tetramethylurea, acetonitrile, nuclease inhibitors such as actin G and cationic salts such as magnesium (Mg²⁺) and lithium (Li⁺) and any of their derivatives. The amount of cationic salt in the composition herein preferably ranges from about 0.1 mM to about 100 mM, and still more preferably from about 0.1 mM to about 10 mM. Viscosity enhancing agents include sodium carboxymethylcellulose, sorbitol, and dextran. The composition can also contain substances known in the art to promote penetration or transport across the blood barrier or membrane of a particular organ e.g., antibody to transferrin receptor. A gel complex of poly-lysine and lactose or poloxamer 407 may be used to facilitate administration in arterial cells.

The pharmaceutical compositions containing the siRNAs, shRNAs or ASOs described herein may be administered to patients in therapeutically effective amounts. As used herein, the term “therapeutically effective amount” refers to an amount sufficient to realize a desired biological effect. For example, a therapeutically effective amount for treating Jordan's Syndrome is an amount sufficient to ameliorate one or more symptoms of Jordan's Syndrome, as described herein (e.g., developmental delay, severe cognitive impairment, ataxic gait, frequent seizures, short attention span, absent speech, intellectual disability, hypotonia, megalencephaly, coordination disorder, seizures and autism).

The appropriate dosage may vary depending upon known factors such as the pharmacodynamic characteristics of the particular active agent, age, health, and weight of the host organism; the condition(s) to be treated, nature and extent of symptoms, kind of concurrent treatment, frequency of treatment, the need for prevention or therapy and/or the effect desired. The dosage will also be calculated dependent upon the particular route of administration selected. Further refinement of the calculations necessary to determine the appropriate dosage for treatment can be made by a practitioner, in the light of the relevant circumstances.

Pharmaceutical compositions herein can be enclosed in ampoules, disposable syringes or multiple dose vials made of glass or plastic. In all cases, the composition should be sterile and should be fluid to the extent that easy syringability exists. It should be stable under the conditions of manufacture and storage and preserved against the contaminating action of microorganisms such as bacteria and fungi. Sterile injectable solutions can be prepared by incorporating the active agent (e.g., infectious particles) in the required amount with one or a combination of ingredients enumerated above, followed by filtered sterilization.

The pharmaceutical compositions herein may be administered by a parenteral route including intravenous, intraarterial, subcutaneous, intraperitoneal or intramuscular injection or infusion, intrathecal or intracranial, e.g., intracerebral or intraventricular, administration. In embodiments, viral particles or pharmaceutical compositions are administered intracerebrally or intracerebroventricularly. In embodiments, the pharmaceutical compositions herein are administered intrathecally.

In embodiments, the pharmaceutical compositions described above are administered to the subject by subcranial injection into the brain or into the spinal cord of the patient or human in need. In embodiments, the use of subcranial administration into the brain results in the administration of the nucleotide sequences described herein directly to brain cells, including glia and neurons. As used herein, the term “neuron” refers to any cell in, or associated with, the function of the brain. The term may refer to any one the types of neurons, including unipolar, bipolar, multipolar and pseudo-unipolar.

TABLE 4

| | PPP2R5D Single Nucleotide | |
|---|---|---|
| Reference Transcript | Polymorphism or Missense Mutation | |
| NM_006245.4 | c.589G>A | p.E197K |
| NM_006245.4 | c.592G>A | p.E198K |
| NM_006245.4 | c.1258G>A | p.E420K |
| NM_006245.4 | c.598G>A | p.E200K |

Nucleotide Sequence Encoding Protein Phosphatase 2 Regulatory Subunit B′Delta (PPP2R5D), mRNA Transcript Variant 1, GenBank Accession NM_006245.4

| (SEQ ID NO: 136) | |
|---|---|
| gcagcgcgca ggcggtggcg aagagacgcc gagcgggccg agtgcggccg agcaaagccg | 60 |
| gagccggagc ggggccgcag gagacgggcc gggtccggac gggccgagat gccctataaa | 120 |
| ctgaaaaagg agaaggagcc ccccaaggtt gccaaatgca cagccaagcc tagcagctcg | 180 |
| ggcaaggatg gtggaggcga gaacactgag gaggcccagc cgcagcccca gccccagccc | 240 |
| cagccccaag cccagtctca gccaccgtca tccaacaagc gtcccagcaa tagcacgccg | 300 |
| ccccccacgc agctcagcaa aatcaagtac tcaggggggc cccagattgt caagaaggag | 360 |
| cgacggcaaa gctcctcccg cttcaacctc agcaagaatc gggagctgca gaagcttcct | 420 |
| gccctgaaag attcgccaac ccaggagcgg gaggagctgt ttatccagaa gctacgccag | 480 |
| tgctgtgtcc tctttgactt cgtgtcagac ccactcagtg acctcaaatt caaggaggtg | 540 |
| aagcgggcag gactcaacga gatggtggag tacatcaccc atagccgtga tgttgtcact | 600 |
| gaggccattt accctgaggc tgtcaccatg ttttcagtga acctcttccg gacgctgcca | 660 |
| ccttcatcga atcccacagg ggctgagttt gacccagagg aagatgagcc caccctggaa | 720 |
| gctgcttggc cacatctcca gctcgtgtat gagttcttct tacgtttcct tgagtctcct | 780 |
| gatttccagc caaacatagc caagaagtac atcgaccaga agtttgtact tgctctccta | 840 |
| gacctatttg acagtgagga tcctcgagag cgggacttcc tcaagaccat tttgcatcgc | 900 |
| atctatggca agtttttggg gctccgggct tatatccgta ggcagatcaa ccacatcttc | 960 |
| tacaggttca tctacgagac ggagcatcac aacgggattg ctgagctcct ggagatcctg | 1020 |
| ggcagcatca tcaatggctt tgccctgccc cttaaagaag agcacaagat gttcctcatc | 1080 |
| cgtgtcctac ttccccttca caaggtcaag tccctgagtg tctaccaccc tcagctggca | 1140 |
| tactgtgtgg tacaattcct ggagaaggag agcagtctga ctgagccggt aattgtggga | 1200 |
| cttctcaagt tttggcccaa gacccacagc cccaaggagg tgatgttctt gaatgagctg | 1260 |
| gaggagattc tggacgtcat tgaaccttct gagttcagca aagtgatgga acccctcttc | 1320 |
| cgccagctgg ccaagtgtgt ctctagcccc catttccagg tggcagagcg tgctctctat | 1380 |
| tactggaaca atgagtacat catgagcctg ataagtgaca atgctgcccg agtcctcccc | 1440 |
| atcatgttcc ctgcactcta caggaactcc aagagccact ggaacaagac aatccatgga | 1500 |
| ctgatctata atgccctgaa gttgtttatg gaaatgaatc agaagctgtt tgatgactgc | 1560 |
| acacaacaat acaaggcaga gaagcagaag ggccggttcc gaatgaagga aagggaagag | 1620 |
| atgtggcaaa aaatcgagga gctggcccgg cttaatcccc agtatcccat gttccgagcc | 1680 |
| cctccaccac tgccccctgt gtactcgatg gagacagaga cccccacagc tgaggacatc | 1740 |
| cagcttctga agaggactgt ggagactgag gctgttcaga tgctaaaaga catcaagaag | 1800 |
| gagaaagtgc tgctgcggag gaagtcggag ctgccccagg acgtgtacac catcaaggca | 1860 |
| ctggaggcgc acaagcgggc ggaagagttc ctaactgcca gccaggaggc tctctgaccc | 1920 |
| ctcacgttcc taccacaggg ccacagccca cacagccctg ggacactgcc ctggccctcc | 1980 |
| atactctgct ccctactggc tgtcttgggg gaaggcagcg cctctctagc tactcaaggg | 2040 |
| agggggatgt gggcacttga agcagggaca cccacagaat ggtccctctt ctccccaaaa | 2100 |
| ggtgttcatg cctccctgtg gctagtacag gctgagcact aagatgctta gtgctcagac | 2160 |
| aacctgggga tgcctgtccc ctacctgctc ctcacccaca gctacctgag gctgctctga | 2220 |
| gaagtacaca caggaataca tacgctcctc tattcttccc ttcatcctca tttgaacgcc | 2280 |
| aggtatctcc cctcctctct ctcccctgca gaggcatgta gggaacagca ggagattatt | 2340 |
| ctcaccaaag ttatgtcaag ccccattggt cccagagtag ctgaagggaa gccaaccccc | 2400 |
| ctgcagcaca aataggcccc ccagtcccag ccgtgtgctg gcagataggg ttgtattatt | 2460 |
| tcttcttacc ccatgcctga ccaagggagg tcaaaaggag aaaagtatag gctgtggaca | 2520 |
| ataactgatg aatatagggc ccagatggac caagtggggc cggggaggga tgaataaaca | 2580 |
| ccctaccccg tgccctgtct ttggtgagca gcagccctgg ggtcacagac atggaaggga | 2640 |
| ccaccctggg gctgactgct tttctgtgct gttggttccc aaaactagaa agaaggaagc | 2700 |
| agggagcggt gccccaagca tggctcctgc caacacctat ttatttcctt gtttgtgcta | 2760 |
| tgctgggcag gccttctctt gtcccttata ggtaccttgg aggggccagg ggctgaggaa | 2820 |
| ggccggaccc aggttccagg ggcgcaggca gtgcggcttt tggctgtgta catagggtgc | 2880 |
| tttattctcc acagagtgat acatgctaag gtgggttggg cttggaccga tgtccccata | 2940 |
| tgtacagaac tgaataaagt gggtctctga gaa | 2973 |

It is understood that the foregoing detailed description and accompanying examples are merely illustrative and are not to be taken as limitations upon the scope of the subject matter described herein, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, compositions, formulations, or methods of use, may be made without departing from the spirit and scope thereof.

What is claimed is:

1. A short interfering ribonucleic acid for inhibiting expression of a mutant allele of the PPP2R5D gene comprising an antisense strand which is complementary to a nucleotide sequence selected from SEQ ID NOs: 1 through 29.

2. The short interfering ribonucleic acid according to claim 1, wherein the mutant allele contains a missense mutation selected from the group consisting of E197K, E198K, E200K and E420K.

3. The short interfering ribonucleic acid according to claim 1, wherein the antisense strand is at least 85%, at least 90%, at least 95%, or 100% complementary to a nucleotide sequence selected from SEQ ID NOs: 1 through 29.

4. The short interfering ribonucleic acid according to claim 1, wherein the antisense strand is selected from SEQ ID NOs: 30-58.

5. The short interfering ribonucleic acid according to claim 1, wherein the short interfering ribonucleic acid further comprises a sense strand, wherein the sense strand is complementary to the antisense strand, and wherein the sense strand comprises a nucleotide sequence selected from SEQ ID NOs: 1 through 29.

6. The short interfering ribonucleic acid according to claim 1, wherein one or more nucleotides contains a modification selected from the group consisting of constrained ethyl bridged nucleic acid (cEt), ethylene-bridged nucleic acid (ENA), 2′-fluoro (2′-F), locked nucleic acid (LNA), 2′-O-methoxyethyl (2′-MOE), 2′-O-methyl (2′-OMe), phosphorodiamidate morpholino oligonucleotide (PMO), peptide nucleic acid (PNA), and phosphorothioate substitution of one or more phosphodiester bonds between nucleotides (PS).

7. The short interfering ribonucleic acid according to claim 1, wherein the 3′ end, the 5′ end or both the 3′ end and the 5′end, are shortened by one, two or three nucleotides.

8. The short interfering ribonucleic acid according to claim 1, further comprising a terminal nucleotide overhang.

9. The short interfering ribonucleic acid according to claim 8, wherein the terminal overhang is dTdT on the 3′ end.

10. A polynucleotide encoding a short interfering ribonucleic acid for inhibiting expression of a mutant allele of the PPP2R5D gene, the short interfering ribonucleic acid comprising an antisense strand which is complementary to a nucleotide sequence selected from SEQ ID NOs: 1 through 29.

11. The polynucleotide according to claim 10, wherein the mutant allele contains a missense mutation selected from the group consisting of E197K, E198K, E200K and E420K.

12. The polynucleotide according to claim 10, wherein the antisense strand is at least 85%, at least 90%, at least 95%, or 100% complementary to a nucleotide sequence selected from SEQ ID Nos: 1 through 29.

13. The polynucleotide according to claim 10, wherein the polynucleotide is selected from SEQ ID NOs: 96 through 124.

14. The polynucleotide according to claim 10, wherein the short interfering ribonucleic acid further comprises a sense strand, wherein the sense strand is complementary to the antisense strand, and wherein the sense strand comprises a nucleotide sequence selected from SEQ ID NOs: 1 through 29.

15. The polynucleotide according to claim 14, wherein the polynucleotide encoding the sense strand is selected from SEQ ID Nos: 67 through 95.

16. The polynucleotide according to claim 10, wherein the short interfering ribonucleic acid is shortened at the 3′ end, the 5′ end or both the 3′ end and the 5′end, by one, two or three nucleotides.

17. The polynucleotide according to claim 10, wherein the short interfering ribonucleic acid further comprises a terminal nucleotide overhang.

18. The polynucleotide according to claim 10, wherein the terminal overhang is dTdT on the 3′ end.

19. A plasmid, viral vector, or viral particle comprising the polynucleotide according to claim 10.

20. A short hairpin ribonucleic acid comprising a first portion, a second portion and a third portion, the first portion comprising any of SEQ ID NOs: 1 through 29, the second portion comprising any of SEQ ID Nos: 59 through 66, and the third portion comprising any of the respective nucleotide sequences complementary to those in SEQ ID NOs: 1 through 29.

21. The short hairpin ribonucleic acid according to claim 20, wherein respective nucleotide sequences comprise any one of SEQ ID NOs: 30 through 58.

22. The short hairpin ribonucleic acid according to claim 20, wherein one or more nucleotides contains a modification selected from the group consisting of constrained ethyl bridged nucleic acid (cEt), ethylene-bridged nucleic acid (ENA), 2′-fluoro (2′-F), locked nucleic acid (LNA), 2′-O-methoxyethyl (2′-MOE), 2′-O-methyl (2′-OMe), phosphorodiamidate morpholino oligonucleotide (PMO), peptide nucleic acid (PNA), and phosphorothioate substitution of one or more phosphodiester bonds between nucleotides (PS).

23. The short hairpin ribonucleic acid according to claim 20, wherein the first portion is shortened at the 3′ end, the 5′ end or both the 3′ end and the 5′end, by one, two or three nucleotides.

24. The short hairpin ribonucleic acid according to claim 20, wherein the third portion is shortened at the 3′ end, the 5′ end or both the 3′ end and the 5′end, by one, two or three nucleotides.

25. A polynucleotide encoding a short hairpin ribonucleic acid, the short hairpin ribonucleic acid comprising a first portion, a second portion and a third portion, the first portion comprising any of SEQ ID NOs: 1 through 29, the second portion comprising any of SEQ ID NOs: 59 through 66, and the third portion comprising any of the respective nucleotide sequences complementary to those in SEQ ID NOs: 1 through 29.

26. The polynucleotide according to claim 25, wherein the polynucleotide encoding the first portion is selected from SEQ ID Nos: 67 through 95.

27. The polynucleotide according to claim 25, wherein the polynucleotide encoding the third portion is selected from SEQ ID Nos: 96 through 124.

28. The polynucleotide according to claim 25, wherein the polynucleotide encoding the second portion is selected from SEQ ID Nos: 59 through 66.

29. A plasmid, viral vector, or viral particle comprising the short hairpin ribonucleic acid according to claim 25.

30. An antisense oligonucleotide targeting a E198K PPP2R5D missense mutation comprising a gapmer oligonucleotide selected from SEQ ID Nos: 125 through 135.

31. The antisense oligonucleotide according to claim 30, wherein the gapmer oligonucleotide is truncated in a 5-10-4, 4-10-4, 4-10-3, 3-10-3, 3-10-2, 5-9-6, 5-9-5, 4-9-5, 4-9-4, 3-9-4, 3-9- 3, 6-8-6, 6-8-5, 5-8-5, 5-8-4, 4-8-4, or 4-8-3 format.


SEQUENCE LISTING (136 sequences)

SEQ ID NO: 1 - RNA, 19 nt, synthetic construct
  misc_feature 1..19: note = Synthetic
  source 1..19: mol_type = other RNA
caaaggaagatgagcccac

SEQ ID NO: 2 - RNA, 19 nt, synthetic construct
  misc_feature 1..19: note = Synthetic
  source 1..19: mol_type = other RNA
aaaggaagatgagcccacc

SEQ ID NO: 3 - RNA, 19 nt, synthetic construct
  misc_feature 1..19: note = Synthetic
  source 1..19: mol_type = other RNA
ccaaaggaagatgagccca

SEQ ID NO: 4 - RNA, 19 nt, synthetic construct
  misc_feature 1..19: note = Synthetic
  source 1..19: mol_type = other RNA
acccaaaggaagatgagcc

SEQ ID NO: 5 - RNA, 19 nt, synthetic construct
  misc_feature 1..19: note = Synthetic
  source 1..19: mol_type = other RNA
cccaaaggaagatgagccc

SEQ ID NO: 6 - RNA, 21 nt, synthetic construct
  misc_feature 1..21: note = Synthetic
  source 1..21: mol_type = other RNA
caaaggaagatgagcccaccc

SEQ ID NO: 7 - RNA, 21 nt, synthetic construct
  misc_feature 1..21: note = Synthetic
  source 1..21: mol_type = other RNA
aaaggaagatgagcccaccct

SEQ ID NO: 8 - RNA, 19 nt, synthetic construct
  misc_feature 1..19: note = Synthetic
  source 1..19: mol_type = other RNA
ccagagaaagatgagccca

SEQ ID NO: 9 - RNA, 19 nt, synthetic construct
  misc_feature 1..19: note = Synthetic
  source 1..19: mol_type = other RNA
cagagaaagatgagcccac

SEQ ID NO: 10 - RNA, 19 nt, synthetic construct
  misc_feature 1..19: note = Synthetic
  source 1..19: mol_type = other RNA
acccagagaaagatgagcc

SEQ ID NO: 11 - RNA, 19 nt, synthetic construct
  misc_feature 1..19: note = Synthetic
  source 1..19: mol_type = other RNA
cccagagaaagatgagccc

SEQ ID NO: 12 - RNA, 21 nt, synthetic construct
  misc_feature 1..21: note = Synthetic
  source 1..21: mol_type = other RNA
agagaaagatgagcccaccct

SEQ ID NO: 13 - RNA, 21 nt, synthetic construct
  misc_feature 1..21: note = Synthetic
  source 1..21: mol_type = other RNA
gagaaagatgagcccaccctg

SEQ ID NO: 14 - RNA, 19 nt, synthetic construct
  misc_feature 1..19: note = Synthetic
  source 1..19: mol_type = other RNA
ccccatttccaggtggcaa

SEQ ID NO: 15 - RNA, 19 nt, synthetic construct
  misc_feature 1..19: note = Synthetic
  source 1..19: mol_type = other RNA
cccatttccaggtggcaaa

SEQ ID NO: 16 - RNA, 19 nt, synthetic construct
  misc_feature 1..19: note = Synthetic
  source 1..19: mol_type = other RNA
catttccaggtggcaaagc

SEQ ID NO: 17 - RNA, 19 nt, synthetic construct
  misc_feature 1..19: note = Synthetic
  source 1..19: mol_type = other RNA
ccatttccaggtggcaaag

SEQ ID NO: 18 - RNA, 21 nt, synthetic construct
  misc_feature 1..21: note = Synthetic
  source 1..21: mol_type = other RNA
ccccatttccaggtggcaaag

SEQ ID NO: 19 - RNA, 21 nt, synthetic construct
  misc_feature 1..21: note = Synthetic
  source 1..21: mol_type = other RNA
tttccaggtggcaaagcgtgc

SEQ ID NO: 20 - RNA, 19 nt, synthetic construct
  misc_feature 1..19: note = Synthetic
  source 1..19: mol_type = other RNA
tttgacccagaggaagata

SEQ ID NO: 21 - RNA, 19 nt, synthetic construct
  misc_feature 1..19: note = Synthetic
  source 1..19: mol_type = other RNA
ttgacccagaggaagataa

SEQ ID NO: 22 - RNA, 19 nt, synthetic construct
  misc_feature 1..19: note = Synthetic
  source 1..19: mol_type = other RNA
tgacccagaggaagataag

SEQ ID NO: 23 - RNA, 21 nt, synthetic construct
  misc_feature 1..21: note = Synthetic
  source 1..21: mol_type = other RNA
tgacccagaggaagataagcc

SEQ ID NO: 24 - RNA, 21 nt, synthetic construct
  misc_feature 1..21: note = Synthetic
  source 1..21: mol_type = other RNA
gacccagaggaagataagccc

SEQ ID NO: 25 - RNA, 21 nt, synthetic construct
  misc_feature 1..21: note = Synthetic
  source 1..21: mol_type = other RNA
acccagaggaagataagccca

SEQ ID NO: 26 - RNA, 19 nt, synthetic construct
  misc_feature 1..19: note = Synthetic
  source 1..19: mol_type = other RNA
gaggaagataagcccaccc

SEQ ID NO: 27 - RNA, 21 nt, synthetic construct
  misc_feature 1..21: note = Synthetic
  source 1..21: mol_type = other RNA
gaggaagataagcccaccctg

SEQ ID NO: 28 - RNA, 21 nt, synthetic construct
  misc_feature 1..21: note = Synthetic
  source 1..21: mol_type = other RNA
aagataagcccaccctggaag

SEQ ID NO: 29 - RNA, 21 nt, synthetic construct
  misc_feature 1..21: note = Synthetic
  source 1..21: mol_type = other RNA
gcccaccctggaagctgcttg

SEQ ID NO: 30 - RNA, 19 nt, synthetic construct
  misc_feature 1..19: note = Synthetic
  source 1..19: mol_type = other RNA
gtgggctcatcttcctttg

SEQ ID NO: 31 - RNA, 19 nt, synthetic construct
  misc_feature 1..19: note = Synthetic
  source 1..19: mol_type = other RNA
ggtgggctcatcttccttt

SEQ ID NO: 32 - RNA, 19 nt, synthetic construct
  misc_feature 1..19: note = Synthetic
  source 1..19: mol_type = other RNA
tgggctcatcttcctttgg

SEQ ID NO: 33 - RNA, 19 nt, synthetic construct
  misc_feature 1..19: note = Synthetic
  source 1..19: mol_type = other RNA
ggctcatcttcctttgggt

SEQ ID NO: 34 - RNA, 19 nt, synthetic construct
  misc_feature 1..19: note = Synthetic
  source 1..19: mol_type = other RNA
gggctcatcttcctttggg

SEQ ID NO: 35 - RNA, 21 nt, synthetic construct
  misc_feature 1..21: note = Synthetic
  source 1..21: mol_type = other RNA
gtgggctcatcttcctttggg

SEQ ID NO: 36 - RNA, 21 nt, synthetic construct
  misc_feature 1..21: note = Synthetic
  source 1..21: mol_type = other RNA
ggtgggctcatcttcctttgg

SEQ ID NO: 37 - RNA, 19 nt, synthetic construct
  misc_feature 1..19: note = Synthetic
  source 1..19: mol_type = other RNA
tgggctcatctttctctgg

SEQ ID NO: 38 - RNA, 19 nt, synthetic construct
  misc_feature 1..19: note = Synthetic
  source 1..19: mol_type = other RNA
gtgggctcatctttctctg

SEQ ID NO: 39 - RNA, 19 nt, synthetic construct
  misc_feature 1..19: note = Synthetic
  source 1..19: mol_type = other RNA
ggctcatctttctctgggt

SEQ ID NO: 40 - RNA, 19 nt, synthetic construct
  misc_feature 1..19: note = Synthetic
  source 1..19: mol_type = other RNA
gggctcatctttctctggg

SEQ ID NO: 41 - RNA, 21 nt, synthetic construct
  misc_feature 1..21: note = Synthetic
  source 1..21: mol_type = other RNA
ggtgggctcatctttctctgg

SEQ ID NO: 42 - RNA, 21 nt, synthetic construct
  misc_feature 1..21: note = Synthetic
  source 1..21: mol_type = other RNA
gggtgggctcatctttctctg

SEQ ID NO: 43 - RNA, 19 nt, synthetic construct
  misc_feature 1..19: note = Synthetic
  source 1..19: mol_type = other RNA
ttgccacctggaaatgggg

SEQ ID NO: 44 - RNA, 19 nt, synthetic construct
  misc_feature 1..19: note = Synthetic
  source 1..19: mol_type = other RNA
tttgccacctggaaatggg

SEQ ID NO: 45 - RNA, 19 nt, synthetic construct
  misc_feature 1..19: note = Synthetic
  source 1..19: mol_type = other RNA
gctttgccacctggaaatg

SEQ ID NO: 46 - RNA, 19 nt, synthetic construct
  misc_feature 1..19: note = Synthetic
  source 1..19: mol_type = other RNA
ctttgccacctggaaatgg

SEQ ID NO: 47 - RNA, 21 nt, synthetic construct
  misc_feature 1..21: note = Synthetic
  source 1..21: mol_type = other RNA
ttgccacctggaaatgggggc

SEQ ID NO: 48 - RNA, 21 nt, synthetic construct
  misc_feature 1..21: note = Synthetic
  source 1..21: mol_type = other RNA
acgctttgccacctggaaatg

SEQ ID NO: 49 - RNA, 19 nt, synthetic construct
  misc_feature 1..19: note = Synthetic
  source 1..19: mol_type = other RNA
tatcttcctctgggtcaaa

SEQ ID NO: 50 - RNA, 19 nt, synthetic construct
  misc_feature 1..19: note = Synthetic
  source 1..19: mol_type = other RNA
ttatcttcctctgggtcaa

SEQ ID NO: 51 - RNA, 19 nt, synthetic construct
  misc_feature 1..19: note = Synthetic
  source 1..19: mol_type = other RNA
cttatcttcctctgggtca

SEQ ID NO: 52 - RNA, 21 nt, synthetic construct
  misc_feature 1..21: note = Synthetic
  source 1..21: mol_type = other RNA
cttatcttcctctgggtcaaa

SEQ ID NO: 53 - RNA, 21 nt, synthetic construct
  misc_feature 1..21: note = Synthetic
  source 1..21: mol_type = other RNA
gcttatcttcctctgggtcaa

SEQ ID NO: 54 - RNA, 21 nt, synthetic construct
  misc_feature 1..21: note = Synthetic
  source 1..21: mol_type = other RNA
ggcttatcttcctctgggtca

SEQ ID NO: 55 - RNA, 19 nt, synthetic construct
  misc_feature 1..19: note = Synthetic
  source 1..19: mol_type = other RNA
gggtgggcttatcttcctc

SEQ ID NO: 56 - RNA, 21 nt, synthetic construct
  misc_feature 1..21: note = Synthetic
  source 1..21: mol_type = other RNA
gggtgggcttatcttcctctg

SEQ ID NO: 57 - RNA, 21 nt, synthetic construct
  misc_feature 1..21: note = Synthetic
  source 1..21: mol_type = other RNA
tccagggtgggcttatcttcc

SEQ ID NO: 58 - RNA, 21 nt, synthetic construct
  misc_feature 1..21: note = Synthetic
  source 1..21: mol_type = other RNA
agcagcttccagggtgggctt

SEQ ID NO: 59-66 - skipped (8 entries)

SEQ ID NO: 67 - DNA, 19 bp, synthetic construct
  misc_feature 1..19: note = Synthetic
  source 1..19: mol_type = other DNA
caaaggaagatgagcccac

SEQ ID NO: 68 - DNA, 19 bp, synthetic construct
  misc_feature 1..19: note = Synthetic
  source 1..19: mol_type = other DNA
aaaggaagatgagcccacc

SEQ ID NO: 69 - DNA, 19 bp, synthetic construct
  misc_feature 1..19: note = Synthetic
  source 1..19: mol_type = other DNA
ccaaaggaagatgagccca

SEQ ID NO: 70 - DNA, 19 bp, synthetic construct
  misc_feature 1..19: note = Synthetic
  source 1..19: mol_type = other DNA
acccaaaggaagatgagcc

SEQ ID NO: 71 - DNA, 19 bp, synthetic construct
  misc_feature 1..19: note = Synthetic
  source 1..19: mol_type = other DNA
cccaaaggaagatgagccc

SEQ ID NO: 72 - DNA, 21 bp, synthetic construct
  misc_feature 1..21: note = Synthetic
  source 1..21: mol_type = other DNA
caaaggaagatgagcccaccc

SEQ ID NO: 73 - DNA, 21 bp, synthetic construct
  misc_feature 1..21: note = Synthetic
  source 1..21: mol_type = other DNA
aaaggaagatgagcccaccct

SEQ ID NO: 74 - DNA, 19 bp, synthetic construct
  misc_feature 1..19: note = Synthetic
  source 1..19: mol_type = other DNA
ccagagaaagatgagccca

SEQ ID NO: 75 - DNA, 19 bp, synthetic construct
  misc_feature 1..19: note = Synthetic
  source 1..19: mol_type = other DNA
cagagaaagatgagcccac

SEQ ID NO: 76 - DNA, 19 bp, synthetic construct
  misc_feature 1..19: note = Synthetic
  source 1..19: mol_type = other DNA
acccagagaaagatgagcc

SEQ ID NO: 77 - DNA, 19 bp, synthetic construct
  misc_feature 1..19: note = Synthetic
  source 1..19: mol_type = other DNA
cccagagaaagatgagccc

SEQ ID NO: 78 - DNA, 21 bp, synthetic construct
  misc_feature 1..21: note = Synthetic
  source 1..21: mol_type = other DNA
agagaaagatgagcccaccct

SEQ ID NO: 79 - DNA, 21 bp, synthetic construct
  misc_feature 1..21: note = Synthetic
  source 1..21: mol_type = other DNA
gagaaagatgagcccaccctg

SEQ ID NO: 80 - DNA, 19 bp, synthetic construct
  misc_feature 1..19: note = Synthetic
  source 1..19: mol_type = other DNA
ccccatttccaggtggcaa

SEQ ID NO: 81 - DNA, 19 bp, synthetic construct
  misc_feature 1..19: note = Synthetic
  source 1..19: mol_type = other DNA
cccatttccaggtggcaaa

SEQ ID NO: 82 - DNA, 19 bp, synthetic construct
  misc_feature 1..19: note = Synthetic
  source 1..19: mol_type = other DNA
catttccaggtggcaaagc

SEQ ID NO: 83 - DNA, 19 bp, synthetic construct
  misc_feature 1..19: note = Synthetic
  source 1..19: mol_type = other DNA
ccatttccaggtggcaaag

SEQ ID NO: 84 - DNA, 21 bp, synthetic construct
  misc_feature 1..21: note = Synthetic
  source 1..21: mol_type = other DNA
ccccatttccaggtggcaaag

SEQ ID NO: 85 - DNA, 21 bp, synthetic construct
  misc_feature 1..21: note = Synthetic
  source 1..21: mol_type = other DNA
tttccaggtggcaaagcgtgc

SEQ ID NO: 86 - DNA, 19 bp, synthetic construct
  misc_feature 1..19: note = Synthetic
  source 1..19: mol_type = other DNA
tttgacccagaggaagata

SEQ ID NO: 87 - DNA, 19 bp, synthetic construct
  misc_feature 1..19: note = Synthetic
  source 1..19: mol_type = other DNA
ttgacccagaggaagataa

SEQ ID NO: 88 - DNA, 19 bp, synthetic construct
  misc_feature 1..19: note = Synthetic
  source 1..19: mol_type = other DNA
tgacccagaggaagataag

SEQ ID NO: 89 - DNA, 21 bp, synthetic construct
  misc_feature 1..21: note = Synthetic
  source 1..21: mol_type = other DNA
tgacccagaggaagataagcc

SEQ ID NO: 90 - DNA, 21 bp, synthetic construct
  misc_feature 1..21: note = Synthetic
  source 1..21: mol_type = other DNA
gacccagaggaagataagccc

SEQ ID NO: 91 - DNA, 21 bp, synthetic construct
  misc_feature 1..21: note = Synthetic
  source 1..21: mol_type = other DNA
acccagaggaagataagccca

SEQ ID NO: 92 - DNA, 19 bp, synthetic construct
  misc_feature 1..19: note = Synthetic
  source 1..19: mol_type = other DNA
gaggaagataagcccaccc

SEQ ID NO: 93 - DNA, 21 bp, synthetic construct
  misc_feature 1..21: note = Synthetic
  source 1..21: mol_type = other DNA
gaggaagataagcccaccctg

SEQ ID NO: 94 - DNA, 21 bp, synthetic construct
  misc_feature 1..21: note = Synthetic
  source 1..21: mol_type = other DNA
aagataagcccaccctggaag

SEQ ID NO: 95 - DNA, 21 bp, synthetic construct
  misc_feature 1..21: note = Synthetic
  source 1..21: mol_type = other DNA
gcccaccctggaagctgcttg

SEQ ID NO: 96 - DNA, 19 bp, synthetic construct
  misc_feature 1..19: note = Synthetic
  source 1..19: mol_type = other DNA
gtgggctcatcttcctttg

SEQ ID NO: 97 - DNA, 19 bp, synthetic construct
  misc_feature 1..19: note = Synthetic
  source 1..19: mol_type = other DNA
ggtgggctcatcttccttt

SEQ ID NO: 98 - DNA, 19 bp, synthetic construct
  misc_feature 1..19: note = Synthetic
  source 1..19: mol_type = other DNA
tgggctcatcttcctttgg

SEQ ID NO: 99 - DNA, 19 bp, synthetic construct
  misc_feature 1..19: note = Synthetic
  source 1..19: mol_type = other DNA
ggctcatcttcctttgggt

SEQ ID NO: 100 - DNA, 19 bp, synthetic construct
  misc_feature 1..19: note = Synthetic
  source 1..19: mol_type = other DNA
gggctcatcttcctttggg

SEQ ID NO: 101 - DNA, 21 bp, synthetic construct
  misc_feature 1..21: note = Synthetic
  source 1..21: mol_type = other DNA
gtgggctcatcttcctttggg

SEQ ID NO: 102 - DNA, 21 bp, synthetic construct
  misc_feature 1..21: note = Synthetic
  source 1..21: mol_type = other DNA
ggtgggctcatcttcctttgg

SEQ ID NO: 103 - DNA, 19 bp, synthetic construct
  misc_feature 1..19: note = Synthetic
  source 1..19: mol_type = other DNA
tgggctcatctttctctgg

SEQ ID NO: 104 - DNA, 19 bp, synthetic construct
  misc_feature 1..19: note = Synthetic
  source 1..19: mol_type = other DNA
gtgggctcatctttctctg

SEQ ID NO: 105 - DNA, 19 bp, synthetic construct
  misc_feature 1..19: note = Synthetic
  source 1..19: mol_type = other DNA
ggctcatctttctctgggt

SEQ ID NO: 106 - DNA, 19 bp, synthetic construct
  misc_feature 1..19: note = Synthetic
  source 1..19: mol_type = other DNA
gggctcatctttctctggg

SEQ ID NO: 107 - DNA, 21 bp, synthetic construct
  misc_feature 1..21: note = Synthetic
  source 1..21: mol_type = other DNA
ggtgggctcatctttctctgg

SEQ ID NO: 108 - DNA, 21 bp, synthetic construct
  misc_feature 1..21: note = Synthetic
  source 1..21: mol_type = other DNA
gggtgggctcatctttctctg

SEQ ID NO: 109 - DNA, 19 bp, synthetic construct
  misc_feature 1..19: note = Synthetic
  source 1..19: mol_type = other DNA
ttgccacctggaaatgggg

SEQ ID NO: 110 - DNA, 19 bp, synthetic construct
  misc_feature 1..19: note = Synthetic
  source 1..19: mol_type = other DNA
tttgccacctggaaatggg

SEQ ID NO: 111 - DNA, 19 bp, synthetic construct
  misc_feature 1..19: note = Synthetic
  source 1..19: mol_type = other DNA
gctttgccacctggaaatg

SEQ ID NO: 112 - DNA, 19 bp, synthetic construct
  misc_feature 1..19: note = Synthetic
  source 1..19: mol_type = other DNA
ctttgccacctggaaatgg

SEQ ID NO: 113 - DNA, 21 bp, synthetic construct
  misc_feature 1..21: note = Synthetic
  source 1..21: mol_type = other DNA
ttgccacctggaaatgggggc

SEQ ID NO: 114 - DNA, 21 bp, synthetic construct
  misc_feature 1..21: note = Synthetic
  source 1..21: mol_type = other DNA
acgctttgccacctggaaatg

SEQ ID NO: 115 - DNA, 21 bp, synthetic construct
  misc_feature 1..21: note = Synthetic
  source 1..21: mol_type = other DNA
ggacttgccggcacccatctg

SEQ ID NO: 116 - DNA, 21 bp, synthetic construct
  misc_feature 1..21: note = Synthetic
  source 1..21: mol_type = other DNA
cttgccggcacccatctgccc

SEQ ID NO: 117 - DNA, 21 bp, synthetic construct
  misc_feature 1..21: note = Synthetic
  source 1..21: mol_type = other DNA
atctgcccataggcgaagatg

SEQ ID NO: 118 - DNA, 20 bp, synthetic construct
  misc_feature 1..20: note = Synthetic
  source 1..20: mol_type = other DNA
cttgccggcacccatctgcc

SEQ ID NO: 119 - DNA, 21 bp, synthetic construct
  misc_feature 1..21: note = Synthetic
  source 1..21: mol_type = other DNA
gacttgccggcacccatctgc

SEQ ID NO: 120 - DNA, 19 bp, synthetic construct
  misc_feature 1..19: note = Synthetic
  source 1..19: mol_type = other DNA
cgcttgctcccagccaggt

SEQ ID NO: 121 - DNA, 19 bp, synthetic construct
  misc_feature 1..19: note = Synthetic
  source 1..19: mol_type = other DNA
ccgcttgctcccagccagg

SEQ ID NO: 122 - DNA, 19 bp, synthetic construct
  misc_feature 1..19: note = Synthetic
  source 1..19: mol_type = other DNA
cccgcttgctcccagccag

SEQ ID NO: 123 - DNA, 19 bp, synthetic construct
  misc_feature 1..19: note = Synthetic
  source 1..19: mol_type = other DNA
tcagcccgcttgctcccag

SEQ ID NO: 124 - DNA, 19 bp, synthetic construct
  misc_feature 1..19: note = Synthetic
  source 1..19: mol_type = other DNA
gagtcagcccgcttgctcc

SEQ ID NO: 125 - RNA, 20 nt, synthetic construct
  misc_feature 1..20: note = Synthetic
  source 1..20: mol_type = other RNA
tctttctctgggtcaaactc

SEQ ID NO: 126 - RNA, 20 nt, synthetic construct
  misc_feature 1..20: note = Synthetic
  source 1..20: mol_type = other RNA
atctttctctgggtcaaact

SEQ ID NO: 127 - RNA, 20 nt, synthetic construct
  misc_feature 1..20: note = Synthetic
  source 1..20: mol_type = other RNA
catctttctctgggtcaaac

SEQ ID NO: 128 - RNA, 20 nt, synthetic construct
  misc_feature 1..20: note = Synthetic
  source 1..20: mol_type = other RNA
tcatctttctctgggtcaaa

SEQ ID NO: 129 - RNA, 20 nt, synthetic construct
  misc_feature 1..20: note = Synthetic
  source 1..20: mol_type = other RNA
ctcatctttctctgggtcaa

SEQ ID NO: 130 - RNA, 20 nt, synthetic construct
  misc_feature 1..20: note = Synthetic
  source 1..20: mol_type = other RNA
gctcatctttctctgggtca

SEQ ID NO: 131 - RNA, 20 nt, synthetic construct
  misc_feature 1..20: note = Synthetic
  source 1..20: mol_type = other RNA
ggctcatctttctctgggtc

SEQ ID NO: 132 - RNA, 20 nt, synthetic construct
  misc_feature 1..20: note = Synthetic
  source 1..20: mol_type = other RNA
gggctcatctttctctgggt

SEQ ID NO: 133 - RNA, 20 nt, synthetic construct
  misc_feature 1..20: note = Synthetic
  source 1..20: mol_type = other RNA
tgggctcatctttctctggg

SEQ ID NO: 134 - RNA, 20 nt, synthetic construct
  misc_feature 1..20: note = Synthetic
  source 1..20: mol_type = other RNA
gtgggctcatctttctctgg

SEQ ID NO: 135 - RNA, 20 nt, synthetic construct
  misc_feature 1..20: note = Synthetic
  source 1..20: mol_type = other RNA
ggtgggctcatctttctctg

SEQ ID NO: 136 - DNA, 2973 bp, Homo sapiens
  misc_feature 1..2973: note = Nucleotide sequence encoding protein phosphatase 2 regulatory subunit B'delta (PPP2R5D), mRNA transcript variant 1, GenBank Accession NM_006245.4
  source 1..2973: mol_type = other DNA
gcagcgcgcaggcggtggcgaagagacgccgagcgggccgagtgcggccgagcaaagccggagccggagcggggccgcaggagacgggccgggtccggacgggccgagatgccctataaactgaaaaaggagaaggagccccccaaggttgccaaatgcacagccaagcctagcagctcgggcaaggatggtggaggcgagaacactgaggaggcccagccgcagccccagccccagccccagccccaagcccagtctcagccaccgtcatccaacaagcgtcccagcaatagcacgccgccccccacgcagctcagcaaaatcaagtactcaggggggccccagattgtcaagaaggagcgacggcaaagctcctcccgcttcaacctcagcaagaatcgggagctgcagaagcttcctgccctgaaagattcgccaacccaggagcgggaggagctgtttatccagaagctacgccagtgctgtgtcctctttgacttcgtgtcagacccactcagtgacctcaaattcaaggaggtgaagcgggcaggactcaacgagatggtggagtacatcacccatagccgtgatgttgtcactgaggccatttaccctgaggctgtcaccatgttttcagtgaacctcttccggacgctgccaccttcatcgaatcccacaggggctgagtttgacccagaggaagatgagcccaccctggaagctgcttggccacatctccagctcgtgtatgagttcttcttacgtttccttgagtctcctgatttccagccaaacatagccaagaagtacatcgaccagaagtttgtacttgctctcctagacctatttgacagtgaggatcctcgagagcgggacttcctcaagaccattttgcatcgcatctatggcaagtttttggggctccgggcttatatccgtaggcagatcaaccacatcttctacaggttcatctacgagacggagcatcacaacgggattgctgagctcctggagatcctgggcagcatcatcaatggctttgccctgccccttaaagaagagcacaagatgttcctcatccgtgtcctacttccccttcacaaggtcaagtccctgagtgtctaccaccctcagctggcatactgtgtggtacaattcctggagaaggagagcagtctgactgagccggtaattgtgggacttctcaagttttggcccaagacccacagccccaaggaggtgatgttcttgaatgagctggaggagattctggacgtcattgaaccttctgagttcagcaaagtgatggaacccctcttccgccagctggccaagtgtgtctctagcccccatttccaggtggcagagcgtgctctctattactggaacaatgagtacatcatgagcctgataagtgacaatgctgcccgagtcctccccatcatgttccctgcactctacaggaactccaagagccactggaacaagacaatccatggactgatctataatgccctgaagttgtttatggaaatgaatcagaagctgtttgatgactgcacacaacaatacaaggcagagaagcagaagggccggttccgaatgaaggaaagggaagagatgtggcaaaaaatcgaggagctggcccggcttaatccccagtatcccatgttccgagcccctccaccactgccccctgtgtactcgatggagacagagacccccacagctgaggacatccagcttctgaagaggactgtggagactgaggctgttcagatgctaaaagacatcaagaaggagaaagtgctgctgcggaggaagtcggagctgccccaggacgtgtacaccatcaaggcactggaggcgcacaagcgggcggaagagttcctaactgccagccaggaggctctctgacccctcacgttcctaccacagggccacagcccacacagccctgggacactgccctggccctccatactctgctccctactggctgtcttgggggaaggcagcgcctctctagctactcaagggagggggatgtgggcacttgaagcagggacacccacagaatggtccctcttctccccaaaaggtgttcatgcctccctgtggctagtacaggctgagcactaagatgcttagtgctcagacaacctggggatgcctgtcccctacctgctcctcacccacagctacctgaggctgctctgagaagtacacacaggaatacatacgctcctctattcttcccttcatcctcatttgaacgccaggtatctcccctcctctctctcccctgcagaggcatgtagggaacagcaggagattattctcaccaaagttatgtcaagccccattggtcccagagtagctgaagggaagccaacccccctgcagcacaaataggccccccagtcccagccgtgtgctggcagatagggttgtattatttcttcttaccccatgcctgaccaagggaggtcaaaaggagaaaagtataggctgtggacaataactgatgaatatagggcccagatggaccaagtggggccggggagggatgaataaacaccctaccccgtgccctgtctttggtgagcagcagccctggggtcacagacatggaagggaccaccctggggctgactgcttttctgtgctgttggttcccaaaactagaaagaaggaagcagggagcggtgccccaagcatggctcctgccaacacctatttatttccttgtttgtgctatgctgggcaggccttctcttgtcccttataggtaccttggaggggccaggggctgaggaaggccggacccaggttccaggggcgcaggcagtgcggcttttggctgtgtacatagggtgctttattctccacagagtgatacatgctaaggtgggttgggcttggaccgatgtccccatatgtacagaactgaataaagtgggtctctgagaa